(19) United States
(12) Patent Application Publication
(10) Pub. No.: US 2002/0005000 A1
(43) Pub. Date: Jan. 17, 2002

(54) Body figure curing slant footwear

(76) Inventor: Byoung-Hoon Choi, Daejeon (KR)

Correspondence Address: BIRCH STEWART KOLASCH & BIRCH, PO BOX 747, FALLS CHURCH, VA, 22040-0747, US

(21) Appl. No.: 09/850,003

(22) Filed: May 8, 2001

(30) Foreign Application Priority Data
Jul. 10, 2000 (KR) 2000-19674

(51) Int. Cl. A43B 7/24; A61F 5/14

(52) U.S. Cl. 36/144; 36/142; 36/25 R

(57) ABSTRACT

The present invention relates to a body figure curing footwear, and more particularly to a body figure curing slant footwear which prevents and cures symptoms such as inwardly curved knee (bow leg) by balancing a body upon walking by slantly forming a part of front portion of footwear sole.

### TECHNICAL FIELD

The present invention relates to a body figure curing slant footwear, and more particularly to a body figure curing slant footwear which prevents and cures symptoms such as inwardly curved knee (bow leg) by balancing a body upon walking by slantly forming a part of front portion of footwear sole.

### BACKGROUND ART

Presently, the footwear is a necessary which could not but be existed for life, and recently a research and development have been made in a view for improving stoutness, convenience and aesthetic sense of footwear. Since a human had started to walk in standing erect state, the foot sole has maintained in parallel to ground surface but center lines of both feet have not maintained in parallel each other whereby widened upon landing of foot sole due to standing erect walking. That is, at a moment taking off the foot sole from ground surface and moving a step, a walking is taken of a form in which front half portion of foot directs to outward about 20°-45° from the walking direction without maintaining straight line toward walking direction, so called out-toed gait (\/) form. In this case, since foot sole surface becomes to touch from outward side, it is leaned toward little toe, so that a case becomes occurred that a balance of whole body is lost. The out-toed gait(bow leg shape) changes normal angle (130°) of heap joint, and femoral region is made to be outwardly turned whereby it makes to be a bow leg shape in which knee is bent as crab leg upon walking.

In accordance with this out-toed gait, since a leg is bent as 0-shape upon walking, it becomes a primary cause shortening a stature (height), and it becomes a cause in which thigh muscle, waist muscle, abdominal muscle, breast muscle and the like are contracted at once. When the stature (height) is shortened upon walking, a balance between horizontal and vertical weights loaded to pelvis becomes broken and a backbone is slowly pressed whereby a phenomenon is occurred in which every backbone joints are twisted, so that human body is changed in form to asymmetric shape of right and left. A twisting of backbone makes a hindrance to nerves derived from backbone being a central nerve within backbone, according to this, nerves hindrance of various organ is induced whereby functional obstacle becomes arisen. Thus, the hindrance of nerves derived from backbone becomes a hindrance cause of human body's physiological function such as respiratory organ or digestive organ or various organ of human body.

Consequently, the out-toed gait brings abnormality of crotch articulation whereby induces pelvis slant, and the pelvis slant brings a difference of foot lengths whereby becoming a cause making the backbone to be bent, and smooth movement (mobility) of crotch articulation and moving range are made to be narrower, and the moving ranges of crossing axis, front and rear axis, and vertical axis are contracted whereby basic two functions of lower limbs, that is, supporting and mobility of body weight can not be smoothly executed. Limitation of crotch articulation's mobility frequently produces a phenomenon which partially blunts the moving of lumber whereby making a paralysis at long calf muscle end of lower limbs and simultaneously spraining the foot, and at the same time becoming to give stress to foot and ankle. Consequently, it is changed to a body form of thigh portion being projected to front-ward and the pelvis becomes drooped, according to this, maintenance of physiological curvature degree of spine is made to be difficult, and front long articulation(front vertebra) of pelvis is changed to a body form of unbalanced type state and developed to spinal abnormal curvature whereby becoming to primary cause producing many serious diseases such as disc or lumbago.

And, since the foot sole is contacted to ground surface from outside whereby foot pressure upon walking is concentrated to outside of foot, a fatigue degree of foot is increased and twisting of toe bone is brought.

Since the primary cause of various problems of human body health as above description is produced from changed gait, an improvement of footwear structure capable of preventing this is necessary.

Heretofore also, there has been offered a footwear which is formed to be inclined to inward side 5 from outward side 4 over whole of sole as shown in FIG. 2 by paying attention to this point (refer to Korean Patent Application 98-6150), but this still had a problem that a slant is given to whole of sole 3 whereby whole of outward side of foot is risen relative to inward side upon walking so that ankle articulation is twisted whereby unreasonableness is given to the ankle and the like.

That is, as much as the bottom surface of foot sole heel has to be vertical with center axis line of shinbone, but since the bottom surface of foot heel and the center axis line of shinbone do not make vertical due to inclination of entire sole whereby be inclined, another problem has been occurred that damage of ankle articulation and knee articulation have been brought.

### OBJECT AND SUMMARY OF THE INVENTION

The present invention is offered by considering such problem as above, and it is an object of the present invention to provide a body figure curing footwear which induces correct walking gait upon walking whereby preventing an inclining of human body as well as fixing and strengthening the muscle and ligament being a part of articulation coupling tissue and at the same time making to evenly contact to ground surface upon walking and grounding of foot sole so that foot pressure can be decreased.

In order to accomplish such object as above, the body figure curing slant footwear in accordance with the present invention is made such that an outward side of front half portion of footwear sole is formed higher as much as 3-10 mm than inward side of it so that a balance of body is taken upon walking whereby symptoms such as bow leg is prevented and cured.

### BRIEF DESCRIPTION OF THE DRAWINGS

FIG. 1 is a perspective view schematically showing a conventional footwear,

FIG. 2 is a view schematically showing a conventional footwear given by slant to whole of sole, and

FIGS. 3(a) to (c) are views schematically showing a body figure curing slant footwear of the present invention.

### DETAILED DESCRIPTION OF PREFERRED EMBODIMENTS

Hereinafter, the present invention will be described more in detail with reference to the accompanying drawings.

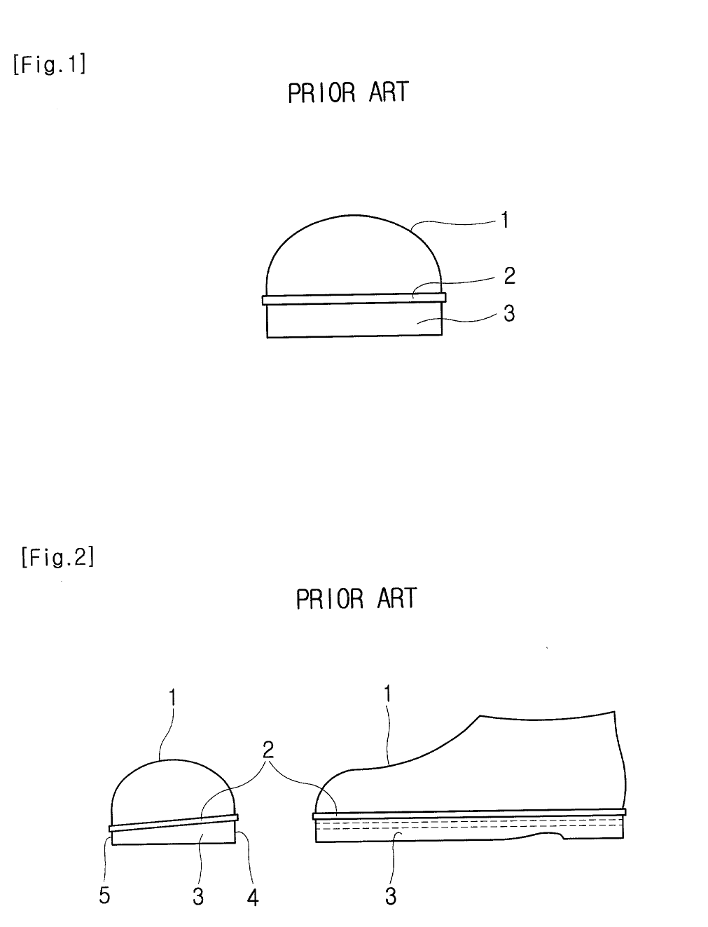

FIG. 1 shows a conventional general footwear which is even in thickness of sole, FIG. 2 shows a cross sectional view of footwear in accordance with prior art, which shows a footwear forming a slant surface to outward side from inward side of foot sole over whole of it from front half portion(toe portion) of sole to rear half portion(toe portion).

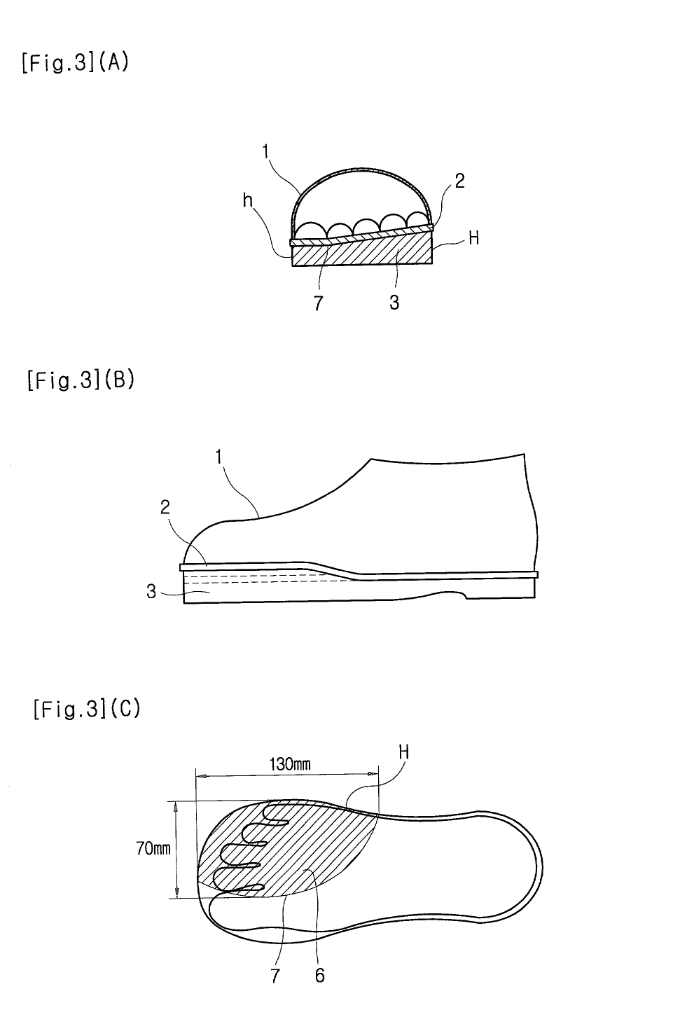

FIGS. 3(a) to (c) are views showing a body figure curing slant footwear in accordance with the present invention, and at only front half portion of the sole, outward side height H is formed higher as much as 3-10 mm than inward side height h. In accordance with the footwear of the present invention according to FIGS. 3, different from conventional slant footwear shown in FIG. 2, only front half portion [heel portion 6 of FIG. 3(c)] of the sole is obliquely formed, so that the outward side of only foot sole front portion is raised relative to the inward side and the out-toed gait is induced to be parallel gait upon stepping the walking forward whereby leg bone is made to be possible to correct to 11 figure type bone from 0 figure type bone, and simultaneously a twisting phenomenon of ankle is eliminated which is a problem of conventional slant footwear appearing at a time when whole of sole is made to be slanted.

The footwear according to the present invention solves a problem of conventional footwear in which a body weight is not loaded to first middle foot bone of big toe side due to so-called out-toed gait upon upright walking or stopping but loaded to fifth middle foot bone of little toe side being an outward side surface of foot sole whereby a deformation is brought to outside leather uppers of footwear and a deformation of toe is brought in severe case. That is, the footwear of the present invention solves a problem of conventional slant footwear, that is, a problem of deformation of ankle articulation appearing at a time when whole of footwear sole is slantly formed.

And, in accordance with the footwear of the present invention, since the footwear sole is made to be horizontal even in case when working by standing long time as wearing the footwear whereby induced to be upright figure, according to this, the vertebra is straightly maintained, and a fatigue feeling is remarkably decreased even in case of standing for long time, and since always correct figure is maintained, a generation of diseases due to incorrect body figure such as waist pain or waist disc articulatory inflammation is prevented.

Reviewing a preferred embodiment of the slant footwear of the present invention, it will be as follows.

In FIGS. 3, figures wearing the footwear of the present invention is shown by dividing respectively to front view (a), side view (b), and plane view (c). (a) shows a state wearing the footwear of the present invention at front, and it is possible to see that fifth middle foot bone side under little toe positioned to outward side is formed higher as much as 3-10 mm than first middle foot bone side under big toe positioned at inward side of the footwear sole. When considering that sole thickness of general footwear is about 8 mm, the height of actual slant becomes about 11 to 20 mm. When viewing a width of general adult's foot is 100 mm and length is 280 mm, a region formed with slant starts to form from about 70 mm area point of fifth middle foot bone, and since its length is placed over about 130 mm range, obliquely lined portion 6 becomes a portion higher than other portion as in FIG. (c). When doing like this, the heel portion of foot is excluded from slant. At this moment, the obliquely lined portion 6 becomes to have a slant which becomes gradually higher as going to outward side H starting from its inward boundary portion 7. Thus, when the outward side of front half portion of footwear becomes higher than the inward side, a walking of the out-toed gait becomes difficult, and naturally the front half portion of foot is moved to inward side whereby a straight line walking is done.

This footwear can be applied to various footwear forms, for instance, casual leather shoes, sports shoes, indoor shoes and the like, as far as it corresponds to substantial object of the present invention saying as body figure curing.

Industrial Applicability

As described above, in accordance with the body figure curing footwear of the invention, there is effect that an outward side height of footwear sole front half portion is formed higher than the inward side height so that a balance of body figure is taken, and a symptoms such as inwardly bent knee (crab leg) is prevented and cured and at the same time a posture is made to be correct upon uprightly standing.

What is claimed is:

1. A body figure curing slant footwear which makes an outward side height of footwear sole front half portion to be higher as much as 3 mm to 10 mm than an inward side height and thereby forming a slant surface between outward side and inward side of footwear front half portion.

2. A body figure curing slant footwear according to claim 1, wherein said slant surface of front half portion of footwear sole is formed to a region reaching at 70 mm to inward direction (big toe direction) from outward end (little toe direction) of footwear sole, and 130 mm to rear end direction from front end portion.
